(19) United States
(12) Patent Application Publication
(10) Pub. No.: US 2009/0105579 A1
(43) Pub. Date: Apr. 23, 2009

(54) METHOD AND APPARATUS FOR REMOTELY CONTROLLED NAVIGATION USING DIAGNOSTICALLY ENHANCED INTRA-OPERATIVE THREE-DIMENSIONAL IMAGE DATA

(76) Inventor: Jeffrey M. Garibaldi, St. Louis, MI (US)

Correspondence Address: Bryan K. Wheelock, Suite 400, 7700 Bonhomme, St. Louis, MO, 63105, US

(21) Appl. No.: 12/250,966

(22) Filed: Oct. 14, 2008

Related U.S. Application Data
(60) Provisional application No. 60/981,472, filed on Oct. 19, 2007.

(51) Int. Cl. A61B 5/05; G06K 9/00; A61B 19/00

(52) U.S. Cl. 600/409; 382/128; 606/130

(57) ABSTRACT

A method of performing intra-operative three-dimensional imaging and registering diagnostic functional information to the three-dimensional anatomical data is introduced. The availability of co-registered diagnostic information to intra-operative data enables fast and efficient navigation to pre-selected target areas, and allows automatic or semi-automatic treatment of cardiac cavity or vascular disease.

### CROSS-REFERENCE TO RELATED APPLICATIONS

This application claims priority to U.S. Provisional Patent Application Ser. No. 60/981,472 filed Oct. 19, 2007. The disclosure of the above-referenced application is incorporated herein by reference.

### FIELD OF THE INVENTION

This invention relates to methods, devices and systems for intra-operative three-dimensional image acquisition, the registration of a sequence of projection images to the three-dimensional reconstructed image data, and the display of diagnostic information registered to the three-dimensional reconstructed image data.

### BACKGROUND OF THE INVENTION

Interventional medicine is the collection of medical procedures in which access to the site of treatment is made by navigation through one of the subject's blood vessels, body cavities or lumens. Interventional medicine technologies have been applied to the manipulation of medical instruments such as guide wires and catheters which contact tissues during surgical navigation procedures, making these procedures more precise, repeatable, and less dependent on the device manipulation skills of the physician. Remote navigation of medical devices is a recent technology that has the potential to provide major improvements to minimally invasive medical procedures. Several presently available interventional medical systems for directing the distal end of a medical device use computer-assisted navigation and a display means for providing an image of the medical device within the anatomy. Such systems can display a projection or cross-section image of the medical device being navigated to a target location obtained from an imaging system such as x-ray fluoroscopy or computed tomography; the surgical navigation being effected through means such as remote control of the orientation of the device distal end and proximal advance of the medical device.

In a typical minimally invasive intervention diagnostic or functional data are collected from a catheter or other interventional devices that are of significant use in treatment planning, guidance, monitoring, and control. For example, in diagnostic applications right-heart catheterization enables pressure and oxygen saturation measure in the right heart chambers, and helps in the diagnosis of valve abnormalities; left-heart catheterization enables evaluation of mitral and aortic valvular defects and myocardial disease. In electrophysiology diagnostic applications, electrical signal measurements may be taken at a number of points within the cardiac cavities to map cardiac activity and determine the source of arrhythmias, fibrillations, and other disorders of the cardiac rhythm. For angioplasty therapeutic applications a number of interventional tools have been developed that are suitable for the treatment of vessel occlusions: guide wires and interventional wires may be proximally advanced and rotated to perform surgical removal of the inner layer of an artery when thickened and atheromatous or occluded by intimal plaque (endarterectomy). Reliable systems have evolved for establishing arterial access, controlling bleeding, and maneuvering catheters and catheter-based devices through the arterial tree to the treatment site.

Fluoroscopic x-ray imaging is the most widely used real-time imaging tool for minimally invasive medical interventions. Fluoroscopy allows immediate visualization of the interventional device progress within the patient's body lumens to the target volume. However significant limitations are associated with the use of x-ray projection imaging. Besides subjecting the patient and potentially the operator to possibly large radiation dose, fluoroscopy is limited by the noisy nature of the acquired images, and by the superimposition of three-dimensional anatomy onto a single plane inherent to projection imaging. The x-ray projection images present shadows of superimposed objects projected onto a single plane. To remedy these limitations, it is common to acquire pre-operative three-dimensional (3D) data by a modality such as computed tomography (CT) or ultrasound. While the pre-operative data provide an excellent 3D anatomical map of the region-of-interest at the time of the data acquisition, and therefore helps in planning the intervention, it is often difficult to register the projection information provided by the fluoroscopy to the pre-operative 3D reconstruction: the patient position with respect to the imaging chain might have changed; organs might have assumed a different shape or relative configuration as compared to the pre-operative acquisition; noise in the images renders the registration and registration evaluation difficult; and real-time demands put strict limits on the amount of computations that might be performed to bring two imaging modalities in registration. Additionally, both the pre-operative 3D CT or ultrasound data and the fluoroscopy images present anatomical information from which diagnostic information might be difficult or impossible to extract; changes due to disease processes might not appear conspicuously on an anatomical map such as provided by x-ray attenuation coefficients that depend mostly on electron density at diagnostic energies. Accordingly there is a need to develop techniques for intra-operative 3D imaging onto which clinical diagnostic data could be co-registered to guide the intervention more effectively and efficiently.

Techniques that have shown potential to help minimally invasive procedures include intra-operative x-ray CT, intra-operative 3D or 4D ultrasound imaging, including intravascular ultrasound (IVUS), optical imaging and optical tissue characterization, and magnetic resonance imaging (MRI). U.S. Pat. No. 6,351,513 issued to Bani-Hashemi et al. and assigned to Siemens Corporate Research, Inc., discloses a method of providing a high-quality representation of a volume having a real-time 3-D reconstruction therein of movement of an object, wherein the real-time movement of the object is determined using a lower-quality representation of only a portion of the volume. In particular U.S. Pat. No. 6,351,513 presents a method of determining the motion of a catheter from a low-quality fluoroscopic image by registering that projection data to a high-quality 3D angiographic reconstruction of the patients vessel. However it does not disclose nor suggest the use of intra-operative 3D data, nor the use of ultrasound imaging, nor the use of two modalities of similar image quality; nor does it teach or suggest the use of magnetic navigation or the co-registration of diagnostic information onto image data. U.S. Pat. No. 6,775,405 issued to Zhu and assigned to Koninkiijke Philips Electronics, N.V., discloses a method of performing image registration of images acquired by different modalities using cross-entropy optimization. U.S. Pat. No. 6,775,405 does not teach nor suggest the use of intra-operative 3D image data, nor does it teach or suggest the co-registration of diagnostic information onto image data.

The present invention addresses the need for intra-operative and preferably real-time 3D imaging of an interventional volume of interest, to which diagnostic and functional information of direct relevance to the intervention can be co-registered to help guide, monitor, and control surgery.

### SUMMARY OF THE INVENTION

One object of the invention is to provide methods, devices and systems to perform a medical procedure utilizing diagnostically enhanced, intra-operative 3D image data set(s), the co-registered intra-operative data and diagnostic information being combined with a virtual or actual image of a remotely controlled navigation device into a real-time display. The 3D image data set can be acquired and reconstructed by various means including 3D X-ray rotational angiography, 3D/4D ultrasound, MRI or other appropriate imaging modality. The 3D reconstructed image data set is registered to the navigation system by various means and approaches depending on the imaging source. For example, a 3D X-ray image can be inherently registered due to a known, fixed mechanical alignment of the X-ray and navigation system, while a 3D ultrasound data set could be registered using a localization system that tracks the position and orientation of the imaging device tip relative to the navigation system. The remotely navigated interventional device is visualized directly by the 3D imaging device (e.g. ultrasound) or indirectly by a localization means and associated device model to derive the virtual appearance of the device in the reconstructed 3D data set. The 3D reconstruction can be a fused representation of the anatomy whereby a static or periodically refreshed volumetric anatomical reconstruction is formed using a sweep of the external or internal imaging device and then fused with a real-time representation of a portion (e.g. a wedge) of the anatomy. The 3D reconstruction presents regions or targets based upon diagnostic and functional information related to the anatomy, the diagnostic information having been acquired through various internal and external methods. For example, the navigation device can be advanced to positions along a vessel or cardiac chamber wall to gather diagnostic information which when processed can then be displayed as regions of activity or therapy targets on the organ wall. The imaging device in this case could be a 3D ultrasound catheter, the catheter location being directly extracted from the image. There are many types of diagnostic information that could be collected including but not limited to voltage, electrical timing, impedance, tissue content and characterization, and blood pressure and velocity. By combining a diagnostically enhanced 3D or 4D reconstructed data set with a rendition of a remotely controlled navigation device that can be displayed directly or virtually co-registered to the 3D or 4D image data, the methods and systems of the present invention enable an operator to efficiently diagnose conditions and deliver correspondingly appropriate therapy to a plurality of targeted points within the patient anatomy.

### BRIEF DESCRIPTION OF THE DRAWINGS

FIG. 1-A shows a patient positioned in a projection imaging system for an interventional procedure such as percutaneous coronary intervention (PCI) and therapy using a controlled minimally invasive modality such as balloon angioplasty;

FIG. 1-B illustrates an interventional device distal end being advanced in the vicinity of a vessel lesion within a theater of intervention such as a coronary artery;

FIG. 2-A shows a patient positioned in a projection imaging system for a minimally invasive procedure such an electrophysiology diagnostic and therapeutic intervention;

FIG. 2-B illustrates an interventional device distal end being navigated through the patient's heart to collect diagnostic information in the left atrium;

FIG. 3 presents a workflow chart for a method of displaying diagnostic data on intra-operative three-dimensional reconstructed data and performing a minimally invasive procedure according to the present invention;

FIG. 4 schematically illustrates co-registered 3D and diagnostic data in a vascular navigation application;

FIG. 5 schematically shows co-registered 3D and diagnostic data in an electrophysiology application;

FIG. 6-A shows an IVUS-enabled catheter being navigated into a heart chamber and acquiring intra-operative ultrasound data; and

FIG. 6-B schematically presents a 3D surface of a heart wall cavity generated from 3D ultrasound data with ECG data superimposed.

Corresponding reference numerals indicate corresponding points throughout the several views of the drawings.

### DETAILED DESCRIPTION OF THE INVENTION

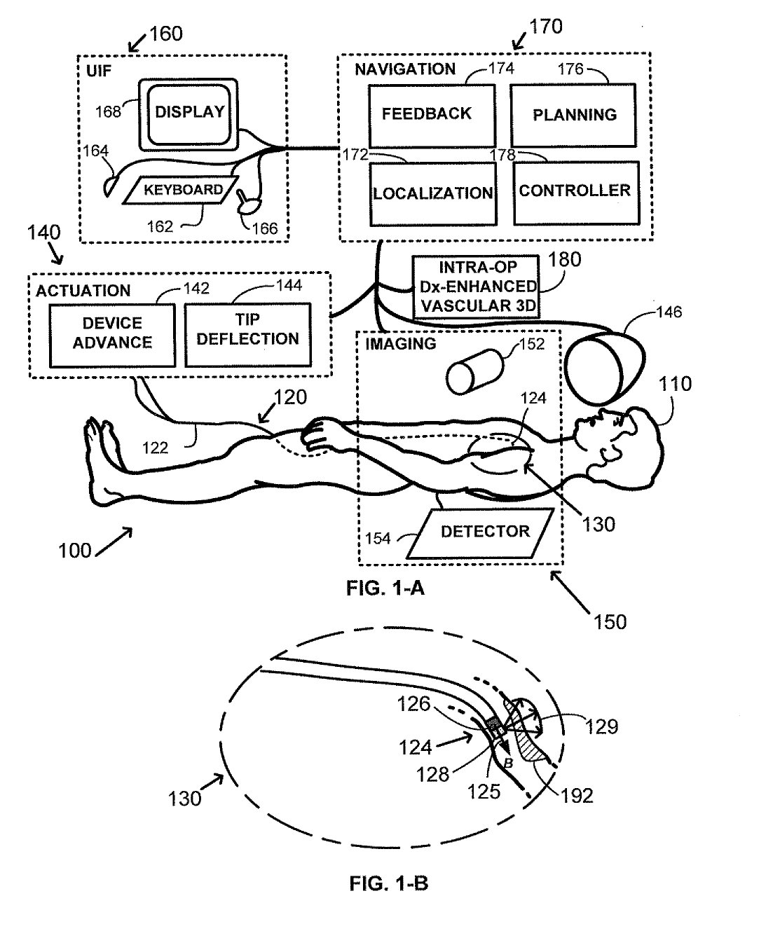

As illustrated in FIG. 1, a patient 110 is positioned within an interventional system, 100. An elongated navigable medical device 120 having a proximal end 122 and a distal end 124 is provided for use in the interventional system 100, FIG. 1-A, and the medical device is inserted into a blood vessel of the patient and navigated to an intervention volume 130. A means of applying force or torque to orient the device distal end 124 is provided, as illustrated by actuation block 140 comprising a device advance/retraction component 142 and a tip deflection component 144. The tip deflection means may be one of (i) a mechanical pull-wire system; (ii) a hydraulic or pneumatic system; (iii) an electrostrictive system; (iv) a magnetic system; or (v) other navigation system as known in the art. For illustration of a preferred embodiment, in magnetic navigation a magnetic field externally generated by magnet(s) assembly 146 orients a small magnet located at the device distal end (126, FIG. 1-B). Real time information is provided to the physician by an imaging sub-system 150, for example an x-ray imaging chain comprising an x-ray tube 152 and an x-ray detector 154, and also possibly by use of a three-dimensional device localization sub-system such as a set of electromagnetic wave receivers located at the device distal end (not shown) and associated external electromagnetic wave emitters (not shown), or other localization device with similar effect such as an electric field-based localization system that is based on sensing an externally applied voltage gradient. In the latter case the conducting body of the wire itself carries the signal recorded by the tip electrode to a proximally located localization system. The physician provides inputs to the navigation system through a user interface (UIF) sub-system 160 comprising user interfaces devices such as a display 168, a keyboard 162, mouse 164, joystick 166, and similar input devices. Display 168 also shows real-time image information acquired by the imaging system 150 and localization information acquired by the three-dimensional localization system. UIF sub-system 160 relays inputs from the user to a navigation sub-system 170 comprising a 3D localization block 172, a feedback block 174, a planning block 176, and a controller 178. Navigation sequences are determined by the planning block 176 based on inputs from the user, possibly pre-operative data and localization data from a localization device and sub-system as described above and processed by localization block 172, and real-time imaging and feedback data processed by feedback block 174; the navigation sequence instructions are then sent to the controller 178 which actuates the interventional device 120 through actuation block 140 to effect device advance and tip deflection. Other navigation sensors might include an ultrasound device, 128 (FIG. 1-B) or other device appropriate for the determination of distance from the device tip to the tissues or for tissue characterization. Further device tip feedback data may include relative tip and tissues positions information provided by a local imaging system, predictive device modeling, or device localization system. In an application to occlusion ablation, additional feedback may be provided by an IVUS device 128 (FIG. 1-B), an optical coherence reflectometry device (not shown), or similar device that allows intravascular and vascular characterization to separately identify plaque or fibrous lesion from vascular wall. In closed loop implementations, the navigation sub-system 170 automatically provides input commands to the device advance 142 and tip orientation 144 actuation components based on feedback data and previously provided input instructions; in semi-closed loop implementations, the physician fine-tunes the navigation control, based in part upon displayed and possibly other feedback data, such as haptic force feedback information. Control commands and feedback data may be communicated from the user interface 160 and navigation sub-system 170 to the device and from the device back to navigation sub-system 170 through cables or other means, such as wireless communications and interfaces. As known in the art, system 100 comprises an electromechanical device advancer 142, capable of precise device advance and retraction based on corresponding control commands.

Sub-system 180 comprises controls and software necessary for the intra-operative acquisition of 3D images and the co-registered superposition of diagnostic and functional information onto the reconstructed 3D image data. In one embodiment of the invention, sub-system 180 processes commands from the user to trigger the acquisition of 3D image data, such as from a computed tomography scanner (not shown) or an IVUS ultrasound device. In one embodiment, an IVUS probe 128 is provided at or near the device distal end 124, and acquires a “wedge” of image data providing information regarding the condition of the vasculature and any existing wall or plaque condition; by rotating interventional IVUS probe 128, either by proximally rotating device 120 or through an IVUS probe rotation means provided within the device itself, a 3D map of ultrasound data may be acquired. The real-time “wedge” data may then be fused onto the 3D intra-operative image data, which in turn may be periodically refreshed by an additional scan image data acquisition. Three-dimensional image data are then processed by sub-system 180 and co-registered to interventional image data provided, for example, by fluoroscopy system 150. Additionally, sub-system 180 interfaces with navigation sub-system 170 such that diagnostic and/or functional information are displayed in co-registered fashion onto the intra-operative 3D data. For example, in electrophysiological applications, electrical activity measured by the interventional device can be displayed in a color rendition onto the 3D data; localization information acquired in real-time, together with co-registration of the interventional device to the imaging system 150 frame of reference, enables real-time display of a real or virtual device image co-registered with the intra-operative 3D image data and then co-registered to the diagnostic information. With respect to the present invention, it is convenient to distinguish intra-operative 3D image data from navigation image data. Although both sets of image data may be acquired by using a similar modality, as for example acquiring 3D intra-operative image data by use of an external probe sweep, and navigation image data by means of an IVUS probe, and although the navigation data may be reconstructed into part of a 3D image data set, the distinction allows separating the 3D image data specifically collected to represent the intra-operative anatomy and super-impose diagnostic data, while the navigation data provides direct and often real-time information with respect to the device distal end position, orientation, and immediate neighborhood. It is understood that implementation wherein both 3D intra-operative data and navigation image data are provided by the same instrument, as for example an external ultrasound system or a CT system, are included within the scope of the present invention.

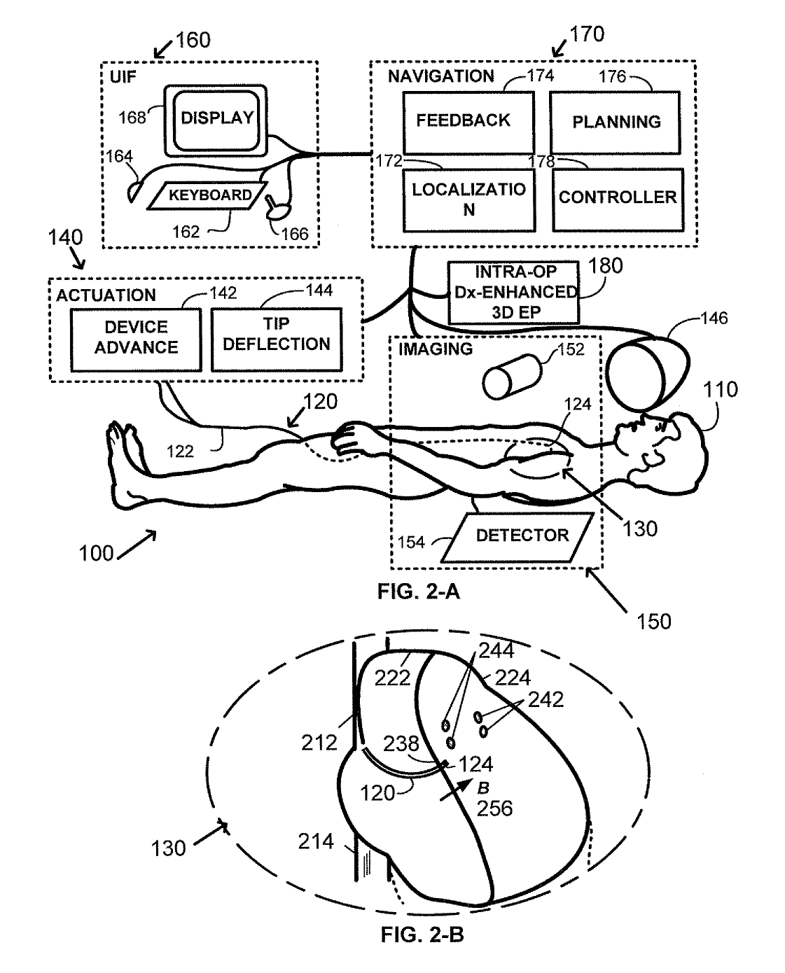

FIG. 2-A presents a patient 110 positioned into an interventional system 100 for an electrophysiology procedure. FIG. 2-B schematically shows the distal end 124 of the interventional device 120 having progressed through the inferior vena cava 214 (or the superior vena cava 212, depending on the application), through the right atrium 222, and through a perforation of the fossa ovalis 238 into the left atrium 224. There the device distal end is magnetically navigated by an externally generated magnetic field B 256 that orients a small magnet positioned at or near the device distal end towards a series of points, for instance associated with the left 242 or right 244 pulmonary arteries. In diagnostic mode, the device collects functional information such as electrical activity. As the device is localized in 3D through localization sub-system 172, the location and orientation of the distal end can be co-registered to 3D anatomical image information, for example acquired by a rotating x-ray fluoroscopy image chain 150 or by a volume CT system (not shown). In such a manner, and after completion of cardiac chamber activity mapping, diagnostic information co-registered to 3D intra-operative image data is immediately available to navigation system 170 to automatically advance the interventional device to a series of points, as determined either by the user or automatically by the navigation system based on prior user inputs. Alternatively to CT or fluoroscopic imaging, externally or internally acquired 3D or 4D ultrasound image data may be used, as known in the art. Through direct image acquisition, or through device tip localization combined with device modeling, an actual or virtual representation of interventional device 120 may be co-registered to the intra-operative 3D image data, showing the location of the device tip with respect to diagnostically identified points targeted for therapy within the reconstructed 3D anatomy.

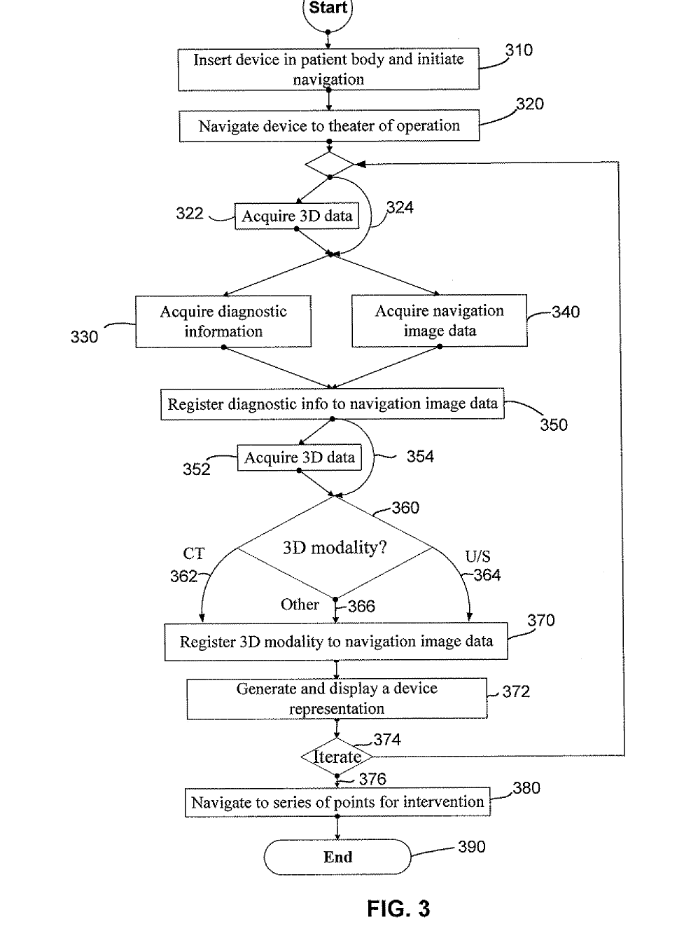

FIG. 3 presents a flow chart for an interventional procedure according to the present invention. At the start of the procedure, 310, an interventional device is inserted into a lumen of a patient, and the device is navigated to a theater of operations, 320. Depending on the intervention workflow, an initial set of intra-operative 3D image data may be acquired, 322, or else the method proceeds directly to the next step, 324. Diagnostic, functional information such as electrical activity of a heart chamber, or plaque characterization in PCI, is then acquired 330, possibly in parallel with navigation image data acquisition, 340. The diagnostic information is then co-registered with the navigation image data, 350; this is accomplished by use of the localization sub-system 172 (FIG. 1-A), through which both the device distal end position and orientation are known with respect to a reference frame of known position and orientation with respect to the navigation imaging system. Depending on the procedure workflow, a first or additional set of three-dimensional image data may be acquired, 352, or else the method proceeds to the next step, 354. For example, and as known in the art, 3D acquisition can be through computed tomography scanning-of the volume of interest, 362. Recently, with the advent of 64 slices CT-systems, considerable interest has been devoted to the application of CT technology to interventional imaging; low-dose imaging modes have been developed whereby both the tube current and tube voltage are modulated as a function of the anatomy from projection to projection, so as to minimize the dose for a level of image quality and image noise. Also, fast image reconstruction techniques, possibly also including ECG cardiac gating, have been developed so that images of acceptable quality are presented to the operator within a minimum delay following acquisition of the last data contributing to the image being reconstructed. Ultrasound is also a modality well suited to the acquisition of intra-operative images: with a fast image refresh rate, no irradiating dose, and a useful cardiac “window” through the chest, ultrasound provides anatomical data that complement x-ray fluoroscopic data when that modality is retained to provide the navigation image data, additionally to providing 3D or 4D volumetric information, 364. Ultrasound technology can also be developed on a small scale, small enough for inclusion of an ultrasound probe at or near the tip of an intra-vascular interventional device. Such a configuration provides advantages as the vessel walls and lumen are imaged at high resolution in real-time. Additionally, other modalities as known in the art, and including optical imaging in various forms, from optical coherence reflectometry to phase tomography, and magnetic resonance imaging, have also been employed to provide 3D image data in intra-operative settings, 366. MRI typically requires a large external system, possibly specifically designed for interventional work to allow relatively easy access to the patient; on the other hand, optical imaging is typically done from within the vessel lumen, as at optical wavelengths photons mean free path is of the order of the millimeter or less. Next, three-dimensional image data are co-registered with the navigation images. Methods to achieve this are known in the art; in the case of x-ray fluoroscopy registration to CT data, it is possible to synthesize a computer-generated projection matching the fluoroscopic projection geometry and techniques by ray tracing through the 3D CT data set; co-registration of two different modalities, such as fluoroscopy and ultrasound, might require specific approaches, such as mutual information, developed for this purpose. Once the navigation image data have been registered to the 3D data, 370, it is then possible to co-register the diagnostic functional information acquired previously to the 3D data, since that information was previously co-registered to the navigation image data in step 350. The functional data are then displayed onto the 3D image data, for instance by mean of colored rendition. Then, in step 372, a device representation is generated and displayed in real-time in co-registration with the 3D image data and the diagnostic data. The device representation may be generated from actual image data, for example acquired from the navigation imaging system, 3D localization data combined with a computer model for the device, or a combination thereof. The steps above may be iterated, depending on the intervention workflow, step 374. At the iteration end, 376, co-registered 3D image data and diagnostic data enable efficient user or automatic navigation of the interventional device shown in real-time in co-registration with the anatomy and the diagnostic information to a series of target points, followed by therapy application (for instance RF ablation) at the identified points. Following therapy performance, the navigation phase of the procedure terminates 390.

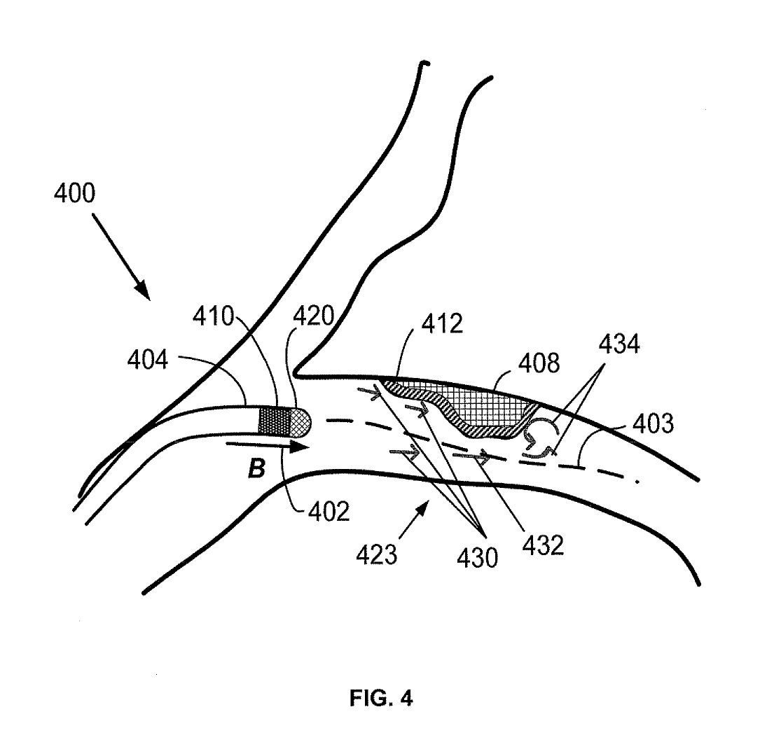

FIG. 4 schematically presents 400 co-registered data for a vascular intervention. In this example the fluoroscopic image shows the vasculature of interest in the neighborhood of the interventional device 404 distal end. A vessel occlusion 408 is also shown with ultrasound imaging and characterization data superimposed, showing in particular the extent of the fibrous cap 412, the volume of the atheromatous plaque 408 representing fatty degeneration of the inner coat of the artery, and also vascular flow vectors 430 indicating the increased blood velocity through the stenosis 423 as well as turbulent flow 434 at the narrowing distal end. FIG. 4 shows the interventional device being advanced for therapy, in the second phase of the procedure following diagnostic data acquisition, co-registration, and display. Device 404 comprises a small magnet 410 suitable for magnetic navigation in an externally generated magnetic field B 402 of less than about 0.1 Tesla, and preferably less than about 0.08 Tesla, and preferably less than about 0.06 Tesla. The device tip 420 is navigated to follow the local vessel lumen 403 and to deliver balloon angioplasty therapy (balloon device not shown). Device tip 420 may comprise further therapy delivery means, such as an antenna for RF ablation, or instrumentation for real-time haemo-dynamic measurements such as blood pressure or velocity. Alternatively FIG. 4 could show a cross-section through a 3D reconstructed CT image data, with co-registered ultrasound diagnostic information being shown super-imposed with a device representation derived from a sequence of real-time fluoroscopic images and a known computer device model.

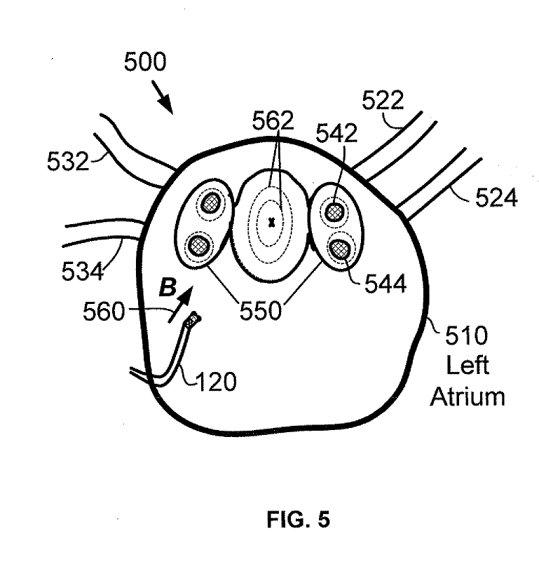

FIG. 5 schematically presents 500 co-registered data for an electrophysiology intervention within the left heart atrium 510. In FIG. 5 appears a 3D anatomical rendition of the left atrium as seen from an anterior-posterior perspective beyond a cut-plane represented by the plane of the figure. Previously acquired electrical signal information, as well as tissue impedance information, have been co-registered with a 3D anatomical map rendition of the left atrium, showing the left superior 532 and inferior 534 and right superior 522 and inferior 524 pulmonary veins ostia. Ablation lines 550 derived from the electrical impedance contours 562 have been automatically computed and are shown in superimposition with the anatomical and electrical information (electrical information not shown in the figure), suggesting treatment target points for RF ablation. Also shown in FIG. 5 is an interventional device 120 being advanced into the left atrium 510 through a perforation of the septal wall. An externally generated magnetic field B 560 orients the device towards the pre-identified lines for RF ablation in the second, therapy, phase of the intervention. The periodic acquisition of projection and/or 3D image data, together with the co-registration of the diagnostic functional information and an image rendition of the interventional device, enables automatic or semi-automatic efficient intervention and treatment of the pre-identified target points or lines.

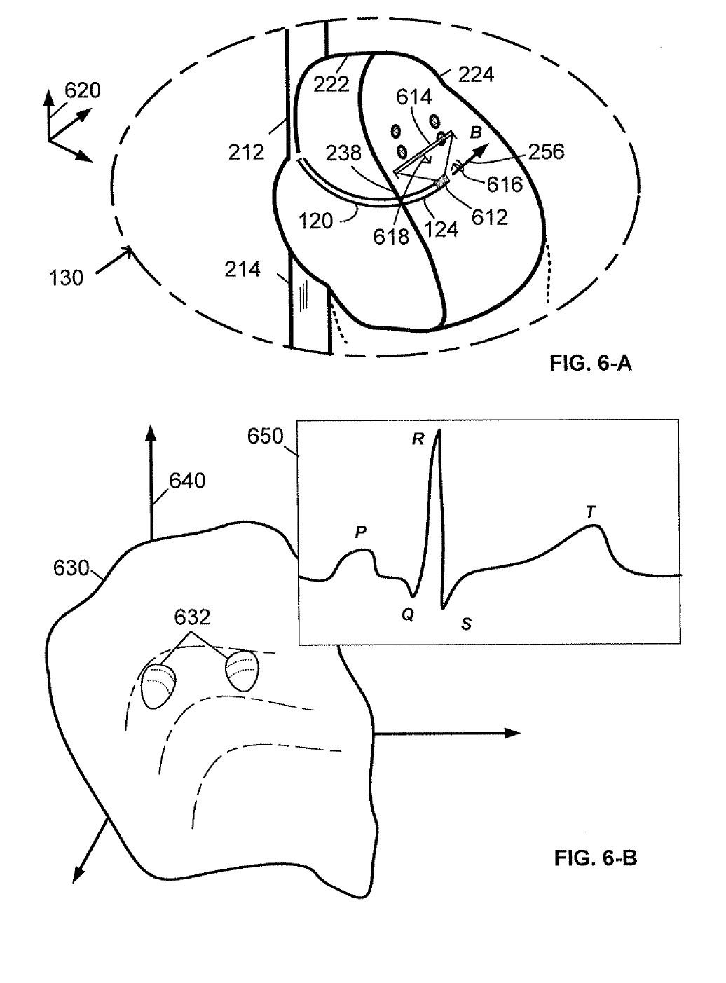

FIG. 6-A presents schematically an IVUS-enabled cardiac catheter 120 being navigated in the left atrium 224 and acquiring sequences of images. Ultrasound probe 612 provided at or near the device distal end 124 is magnetically navigated by externally generated magnetic field B 256. A fan of ultrasound waves is emitted and received by probe 612 and image data reconstruction leads to the generation of image data on a sector or wedge 614. Means provided for rotating the ultrasound probe with respect to local device longitudinal axis 616 (shown superimposed with the magnetic field 256) enables motion of the fan with respect to the anatomy in a direction 618 perpendicular to the wedge plane. FIG. 6-B shows a three-dimensional heart surface 630 reconstructed from the IVUS-acquired ultrasound data, and registered to known interventional system reference frame 640. The surface is periodically refreshed by fusing the most recently acquired wedge of ultrasound data to the representation previously developed. In the situation described in FIG. 6-B, the lower pulmonary veins ostia 632 have just been imaged by the ultrasound beam and the corresponding surface data updated in surface representation 630. Further, and ECG trace data 650 is shown as part of the display, possibly also indicating through coloring of a time range the ECG interval during which the latest wedge data were acquired.

Many other situations where co-registered diagnostic information presented on intra-operative 3D data will help improve intervention efficiency, success rates, and eventually patient outcomes, are not illustrated but are within the scope of this invention. For example, the intra-operative image data could be 3D or 4D; with a periodic 3D image data refresh, either driven by a predetermined time schedule or by intervention-specific events, such as the progress of the interventional device to pre-determined anatomical features or tissue targets; or changes in monitored diagnostic information. Availability of at least one 3D intra-operative data set ensures that better morphological information is obtained as compared to any pre-operative data acquired by a similar procedure. Data set matching and co-registration is aided by effective localization tools, as match image measures tend to be evaluated in a smaller neighborhood of the optimum, and therefore many local extrema in the registration algorithm may be avoided. For illustration, image matching techniques have been previously developed to co-register and co-represent ultrasound image frames acquired by a moving probe in an extended, seamless field of view: in this setting, the problem reduces to that of finding the similarity transformation (parameters: translation, rotation, scaling) that minimizes the mean-squared error between candidate match points; other image measures may include the minimum of the sum of absolute differences or similar mathematical distance measures. While registration methods of images from a similar modality, such as x-ray fluoroscopy projections to CT image data or ultrasound frame to frame have been known in the art for more than a decade, more recently specific techniques such a mutual image information have been proposed to effect co-registration of images acquired by different modalities. Mutual information or relative entropy measures the statistical dependence or information redundancy between the image intensities of corresponding voxels in both images, which is assumed to be maximal if the images are geometrically aligned. Initial results indicate that sub-voxel accuracy may be achieved completely automatically and without any prior segmentation, feature extraction, or other preprocessing steps.

Further, it is understood that a wide range of diagnostic functional information may be acquired in minimally invasive procedures and might be available to guide an intervention to specific target points representative of various types of dysfunctions. Electrophysiology depends critically on electrical mapping of the heart to determine areas of abnormally placed secondary pacemaker driving the heart at a higher rate than normal, re-entry circuits, or heart blocks. Arrhythmias can originate from an ectopic focus or center that may be located at any point within the heart. Disturbances in the cardiac rhythm also originate from the formation of a disorganized electrical circuit, called “re-entry” and resulting in a reentrant rhythm, usually located within the atrium, at the junction between an atrium and a ventricle, or within a ventricle. In a reentrant rhythm, an impulse circulates continuously in a local, damaged area of the heart, causing irregular heart stimulation at an abnormally high rate. Finally various forms of heart blocks can form, preventing the normal propagation of the electrical impulses through the heart, slowing down or completely stopping the heart. Heart blocks originate in a point of local heart damage, and can be located within a chamber, or at the junction of two chambers. The determination of tissue impedance as a guide to tissue ablation, and particularly left atrium ablation around the pulmonary vein ostia, has been shown to be of significant help in guiding the procedure and ensuring a higher success rate. In PCI applications, classification of plaque as for example using ultrasound imaging or optical imaging or characterization, are known to be predictor of interventional success.

Although the method has been illustrated for magnetic navigation applications, it is clear that it may also be applied in conjunction with other means of navigation. For example, the navigation means may comprise mechanical actuation, as per use of a set of pull-wires that enable distal device bending, by itself or in conjunction with proximal device advance and rotation. The navigation means may also comprise other techniques known in the art, such as electrostrictive device control. Further navigation means may comprise combination of the above methods, such as combination of magnetic and electrostrictive navigation, combination of mechanical and electrostrictive navigation, or combination of magnetic and mechanical navigation.

The advantages of the above described embodiments and improvements should be readily apparent to one skilled in the art, as to enabling intra-operative three-dimensional data acquisition and display, display of diagnostic or functional information co-registered to the three-dimensional intra-operative data, and real-time display of an actual or virtual image of the interventional device co-registered with the three-dimensional anatomical image showing diagnostic information. Additional design considerations may be incorporated without departing from the spirit and scope of the invention. Accordingly, it is not intended that the invention be limited by the particular embodiment or form described above, but by the appended claims.

What is claimed is:

1. A method of navigating an interventional device to a set of target points during an interventional procedure, the method comprising:
(i) acquiring at least one set of three-dimensional image data during the procedure;
(ii) reconstructing the three-dimensional data;
(iii) dynamically acquiring a series of images showing at least part of the interventional device;
(iv) advancing the interventional device to a set of target points identified on the at least one set of three-dimensional image data of step (i);
(v) collecting diagnostic information on at least one sub-set of the set of target points of step (iv);
(vi) registering the diagnostic information of step v) to the series of images of step (iii);
(vii) registering at least part of the series of images of step iii) to the at least one set of three-dimensional image data;
(viii) registering the diagnostic information of step v) to the at least one set of three-dimensional image data; and
(ix) guiding the interventional procedure to perform therapy on at least one subset of the set of target points of step iv) using the at least one co-registered set of three-dimensional image data and diagnostic data.

2. The method according to claim 1, further comprising displaying a virtual representation or actual image of the interventional device derived from image data or device model co-registered with the at least one co-registered set of three-dimensional image data and diagnostic data.

3. The method according to claim 1, wherein the collected diagnostic information of step (v) is at least one of the group consisting of (a) electrical activity data; (b) tissue characterization data; (c) tissue electrical impedance data; (d) blood pressure; (e) blood velocity; (f) blood oxygen saturation; and (g).

4. The method according to claim 3, wherein the tissue characterization is performed by optical methods or ultrasound imaging.

5. The method according to claim 11 wherein the navigating is performed using a least one of the group of methods comprising (a) magnetic navigation; (b) mechanical navigation; and (c) electrostrictive navigation.

6.-11. (canceled)

12. A system for automatic or semi-automatic guidance of a remotely controlled interventional device in a patient's body lumens, the system comprising:
(i) means for acquiring three-dimensional image data;
(ii) reconstructing three-dimensional image data;
(iii) advancing and orienting the interventional device distal end;
(iv) collecting diagnostic information at a set of points through the interventional device;
(v) identifying target points on three-dimensional image data;
(vi) acquiring a sequence of images;
(vii) registering the sequence of images to three-dimensional image data;
(viii) registering diagnostic information to three-dimensional data; and
(ix) guiding the remotely controlled interventional device to a target point based on co-registered diagnostic information on three-dimensional image data.

13. The system of claim 12, further comprising means for the generation of a representation of the interventional device.

14. The system of claim 13, wherein the interventional device representation is obtained from image data or from an interventional device model, or a combination thereof.

15. The system of claim 13, further comprising means for displaying the device representation in co-registration with three-dimensional image data and diagnostic information.

16. The system of claim 15, wherein means for displaying the device representation comprises means for updating the co-registered display within 1 second of any change in the position of the device with respect to the patient's body lumens.

17. The system of claim 16, further comprising means for the automatic detection of changes in the position of the device with respect to the patient's body lumens.

18.-26. (canceled)

27. A method of displaying physiologic information about an operating region in a subject, the method comprising:
imaging the operating region in a subject;
while imaging the operating region, navigating a mapping catheter to a plurality of mapping sites in the operating region, and using the mapping catheter to measure a physiologic property at each mapping site;
displaying an image of the operating region; and
displaying indicators of the measured physiologic property on the displayed image of the operating region, at positions on the displayed image of the operating region corresponding to the mapping sites at which the physiologic property was measured.

28.-30. (canceled)

31. The method of claim 27 further comprising determining the position of each mapping site on the displayed image by processing imaging data of the operating region and mapping catheter at each of the mapping sites.

32. The method of claim 27 wherein the mapping catheter is navigating using a remote navigation system that is one of a magnetic navigation system or a mechanical robotic tem.

33.-34. (canceled)

35. The method of claim 27 wherein the indicators include color coded portions of the image.

36. The method of claim 27 wherein the indicators include symbols.

37. The method of claim 27 wherein the indicators include numeric values.

38. The method of claim 27 wherein the imaging is ultrasonic imaging.

39. The method of claim 27 wherein the physiologic property is an electrical signal

40. The method of claim 27 wherein the electrical signal is an ECG signal.

41.-71. (canceled)
